(19) United States
(12) Patent Application Publication
(10) Pub. No.: US 2022/0054233 A1
(43) Pub. Date: Feb. 24, 2022

(54) DENTAL DEVICE

(71) Applicant: Ryan Stainsby, Darlington (GB)

(72) Inventor: Ryan Stainsby, Darlington (GB)

(21) Appl. No.: 17/408,757

(22) Filed: Aug. 23, 2021

(30) Foreign Application Priority Data
Aug. 24, 2020 (GB) 2013182.7
Jan. 13, 2021 (GB) 2100436.1

(51) Int. Cl. A61C 7/00; A61C 7/12; G16H 40/67

(52) U.S. Cl. CPC A61C 7/002; G16H 40/67; A61C 7/12

(57) ABSTRACT

A dental device comprising one or more elongate elements such as wires arranged to contact and exert pressure on one or more teeth, and a reel, which reel is arranged to move the elongate element.

A method or apparatus for controlling movement of one or more teeth comprising a device and a software application operated remotely.

### CROSS-REFERENCE TO RELATED APPLICATIONS

The present application claims priority to GB Application No. 2013182.7, entitled “A dental device”, filed 24 Aug. 2020; and GB Application No. 2100436.1, entitled “A dental device”, filed 13 Jan. 2021—PDAS CODE B08D, the contents of which is hereby incorporated by reference herein in its entirety.

### FIELD OF THE INVENTION

The present invention relates to a dental device, in particular a device for providing and controlling movement of teeth; more particularly but not exclusively for orthodontists or similar specialists and their clients or device users.

### BACKGROUND AND PRIOR ART

Increasing awareness of cosmetic dentistry is a factor in many societies, leading to a proliferation of orthodontists and other specialists in these fields, as techniques and markets have improved.

Frequently in order to rearrange and straighten or correct teeth it can be necessary to wear braces and retainers and tracks, which act to gradually move the teeth over a long period of time to correct and improve their positions in a user's mouth.

This process is time-consuming and can additionally require the user to attend an orthodontist or specialist with frequent check-ups and adjustments made to the wires that are accomplishing the movement of the teeth.

This is resource inefficient both for the user and the orthodontist or specialist dentist.

The present invention arose in order to overcome problems suffered by existing devices.

### SUMMARY OF THE INVENTION

According to a first aspect of the invention there is provided a dental device for location in a user's mouth comprises at least one elongate element which is arranged to exert a force on one or more teeth and a means for retracting and/or extending the at least one elongate element in order to manipulate the one or more teeth.

Preferably, the at least one elongate element is selected from the group comprising: a wire; a band; a chain; a coil spring and a ligature.

Preferably, the means for retracting or extending the at least one elongate element is selected from the group comprising: a linear actuator; a lever and a reel

The linear actuator may include a selected one of: a piston and cylinder; a rack and pinion; a worm drive; and contra-rotating rollers engaging the elongate element therebetween.

The means for retracting or extending the at least one elongate element may comprise a lever, the lever being arranged for rotation about an axis and the elongate element attached to the lever at a point distal from the axis.

Whilst a number of mechanisms for causing retraction and/or extension of the at least one elongate element have been described, the invention is not restricted to the described mechanisms. Any mechanism capable of causing retraction or extension of the at least one elongate element will suffice.

Advantageously, the at least one elongate element is arranged to exert a force on one or more teeth by either contacting one or more teeth or engaging with a member such as a pad or similar which may be attached to, onto or around one or more teeth.

The member attached to said one more teeth may a selected one of: a pad; a bracket and a band.

In some embodiments the device comprises a plurality of wires, arranged to contact one or more teeth, so as to act to alter positioning of the tooth or teeth.

The at least one elongate element may be provided by a plurality of elongate elements.

In some embodiments the means for retracting and/or extending the at least one elongate element comprises a motor, which motor may be adapted to move the at least one elongate element, such as a wire or wires. Where the means for retracting and/or extending the at least one elongate element includes a reel the motor may drive or move the reel, for example shortening the wire(s) by turning the reel.

The means for retracting and/or extending the at least one elongate element may comprise a motor, the motor configured to power the means for retracting and/or extending the at least one elongate element

The means for retracting and/or extending the at least one elongate element may be comprised in an elongate element movement module. Said module may be controlled by a local and/or remote control means. Such remote control means may be envisaged to be wireless, wherein the module may comprise a wireless transceiver.

The at least one elongate element may engage in an elongate element movement module.

The module may include a transceiver for wireless connection to a remote controller.

In some embodiments the wireless transceiver may be envisaged to operate with inter-machine operability such as Bluetooth®. In other embodiments wireless connectivity may be provided by shortwave radio signals, infrared or other local or remote transmittable connectivity.

In some embodiments the module may comprise a socket, for example for connection of a wired connection, such as for example so as to allow data and/or electrical energy transmission.

The device may further comprise a source of electrical energy.

For example the device may comprise a battery, which may be rechargeable. In other embodiments, in addition or the alternative, the battery may be displaceable and/or disposable.

In some embodiments the device may comprise one or more sensors, which sensors may be envisaged to monitor conditions associated with the at least one elongate element and/or tooth or teeth. For example the module may comprise a sensor adapted to measure force acting on the at least one elongate element.

The device may comprise at least one sensor, the at least one sensor configured to monitor a condition associated with the at least one elongate element and/or tooth or teeth.

The at least one sensor may be configured to sense the force exerted on the at least one elongate element and/or tooth or teeth and/or the distance moved by the at least one elongate element and/or tooth or teeth.

The device may include a processor configured to control and/or recognise movement of the tooth or teeth and/or the at least one elongate element and the processor may be comprised in the module.

By providing means to control and/or recognise movement of the teeth and/or the at least one elongate element, for example by monitoring force acting on the at least one elongate element the orthodontist can monitor both the positioning of the teeth and the user's experience of the teeth movements, and may be enabled to both control and reflect the experience according to information acquired. For example, by monitoring how quickly the teeth move after adjustment of the at least one elongate element, the orthodontist may adjust the degree of adjustment of the at least one elongate element on each occasion of adjustment.

Orthodontists and users can then see the progress of treatment via software that may be installed on a plurality of different electronic items of equipment such as personal computers or smartphones.

This software may be programmed to generate indicia showing how far each tooth has moved and then the orthodontist can adapt as necessary. This advantageously means faster and more precise results for the patient.

In some embodiments the processor may comprise a microcontroller, which may include a timer, wherein the module may be configured to adjust the force acting on the at least one elongate element according to time of day, for example to increase force over a period of time incrementally and/or adjust for waking hours. Adjusting the force acting on the at least one elongate element may include a force release feature.

In some embodiments the micro-controller may be configured to control force acting on the at least one elongate element according to preferences that may be set by the user or orthodontist as appropriate. In other embodiments the user may be provided with an override option for use as required for example. An override feature may be useful during the period following adjustment of the at least one elongate element by the orthodontist. Users can experience moderate to severe pain in the days immediately following an adjustment (decrease in length and increase in force of the at least one elongate element). It would be useful to provide a means whereby the user may relax the adjustment for a period of time. A user may elect to relax tensioning in the at least one elongate element overnight, in order to be able to sleep for example, or on a more regular cyclic basis, in order to obtain pain relief. For example the micro controller could be programmed or otherwise caused to relax the force on the at least one elongate element for a period of 1 hour and apply the new force (associated with the new length of the at least one elongate element) for a period of 3 hours. This may be done over a period of a short number of days, for example four days. The duration of each part of the cycle may be changed in order to accustom the user to a new length of the at least one elongate element. For example, immediately after adjustment the period may be 1 hour at the new length/force followed by 1 hour relaxed to the old force; followed by a period where the at least one elongate element is set to the new force for 1.5 hours, followed by 1 hour at the relaxed force; followed by 2 hours at the new force followed by 1 hour at the related force.

The processor includes a timer, and wherein the module is configurable to adjust the force acting on the at least one elongate element

The device may further include a force release and/or force reduction means

The force release and/or reduction means may be commanded by the processor.

The device may further comprise an elongate element lock, which may include an elongate element gripper.

In the above-mentioned scenarios, the relaxed force is not less than the force acting on the at least one elongate element immediately preceding the adjustment.

In some embodiments the device may include a clutch mechanism. Such a clutch mechanism may relieve the motor from force of the at least one elongate element, for example a gripper preventing return of the at least one elongate element until a solenoid valve release clutches for example.

In some embodiments the motor may be geared to increase torque and slow motion. It may be envisaged in use the device is maintained within a mouth. In this way it may be envisaged that the device may comprise a user attachment means. Such attachment means may comprise the wire or wires, or may comprise a separate attachment means. This attachment means may comprise adhesive for example.

The means for retracting and/or extending the at least one elongate element may comprise a manually operable mechanism, which may be comprised in the module. The module may include the means to adjust the length of the at least one elongate element manually. This may be desirable as a back up mechanism if the battery fails for example. The manual adjustment means may comprise a screw or pin engage able by a tool to generate rotation. Such rotation may drive the means for retracting or extending the at least one elongate element.

The module may be attached to one of the teeth. This may be achieved with a suitable adhesive or bands that fit around one or more teeth, typically to the rear of the user's mouth.

The module may be adapted for fixed attachment to a tooth or teeth.

In another embodiment the module is removable from the user's mouth, and the device includes a module mount attached to one or more of the user's teeth. The module is attached to the mount to affect a change in length of the at least one elongate element and is removed after the change has been effected. In such an arrangement, the mount includes a force maintaining means which holds the at least one elongate element in the position to which it was moved by the tensioner of the module. Such a force maintaining means may comprise a gripper arranged to engage with the at least one elongate element and which facilitates movement of the at least one elongate element in one direction (shortening of the at least one elongate element) and which resists movement in the opposite direction. The gripper may comprise a cam and/or a hinged element and/or teeth oriented in a particular direction; and/or an element that is deformable in one direction and which resists deformation in another direction.

The module may be adapted for removable attachment to a tooth or teeth, the tooth or teeth having a bracket fixed thereto, the module removably attachable to the bracket.

According to a second aspect of the present invention there is provided an apparatus for controlling movement of one or more teeth comprising a device substantially as described herein and a software application operated remotely.

According to a third aspect of the invention there is provided a method for controlling movement of one or more teeth comprising an apparatus according to the second aspect of the invention wherein the device thereof is attached to teeth of a user and wherein the software application is operated at least periodically to adjust the length of the at least one elongate element.

Preferably, the software application is operated remotely.

The software application may monitor the device substantially continuously or periodically between adjustments of the length of the at least one elongate element.

### BRIEF DESCRIPTION OF DRAWINGS

In the Drawings, which illustrate preferred embodiments of a device of the invention and which are by way of example:

FIG. 1 shows an isometric view of an embodiment of a module for the device according to the invention;

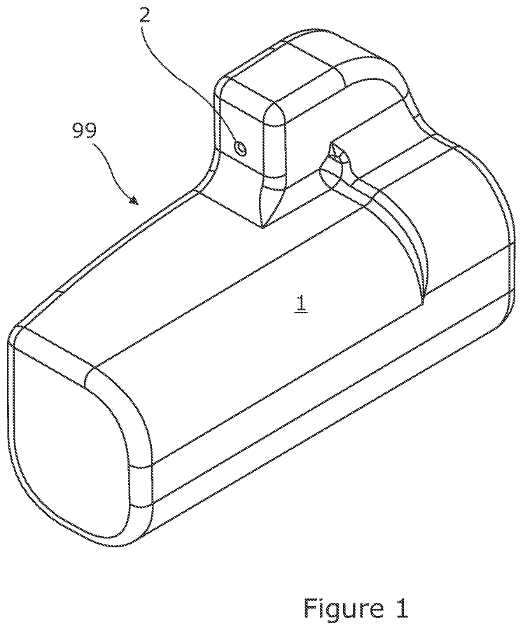

FIG. 2 shows a reverse isometric view of the embodiment of the module shown in FIG. 1;

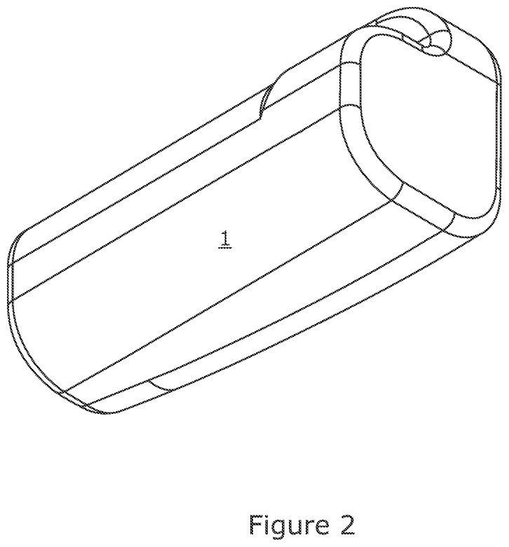

FIG. 3 shows an exploded isometric view of the embodiment of the module shown in FIG. 1;

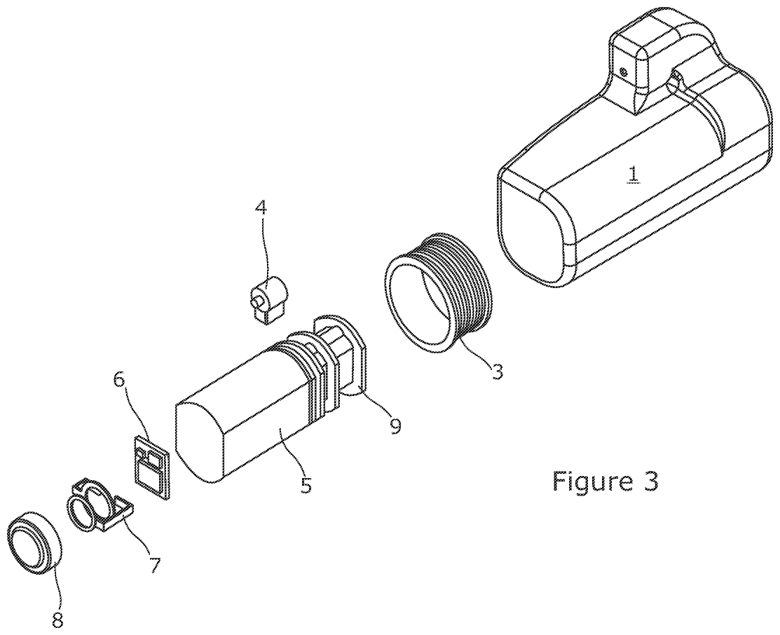

FIG. 4 shows a reverse exploded isometric view of the embodiment of the module shown in FIG. 1;

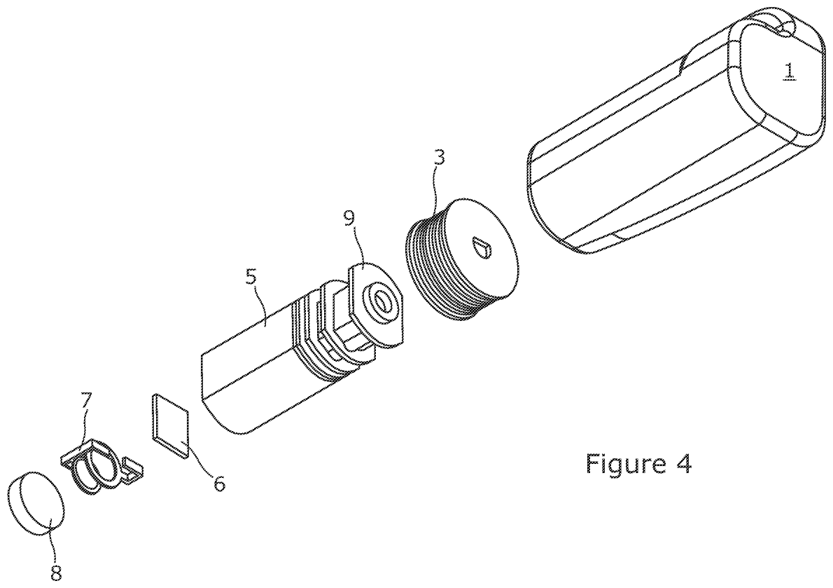

FIG. 5 shows a diagrammatic sketch of a view of the embodiment of the device in use on a mouth, with a wire;

FIG. 6 illustrates an embodiment of the invention where the module is removable from the user's mouth;

FIGS. 7a to 7f illustrate alternative mechanisms for retracting or extending the elongate element of the device of the invention;

FIG. 8 is a block diagram illustrating the electronic system of the module;

FIG. 9 illustrates a smart phone programmed with the software of the invention;

FIG. 10 illustrates a computer device operable by an orthodontist; and

FIG. 11 illustrates a computer device operable by a patient.

### DETAILED DESCRIPTION OF FIGURES

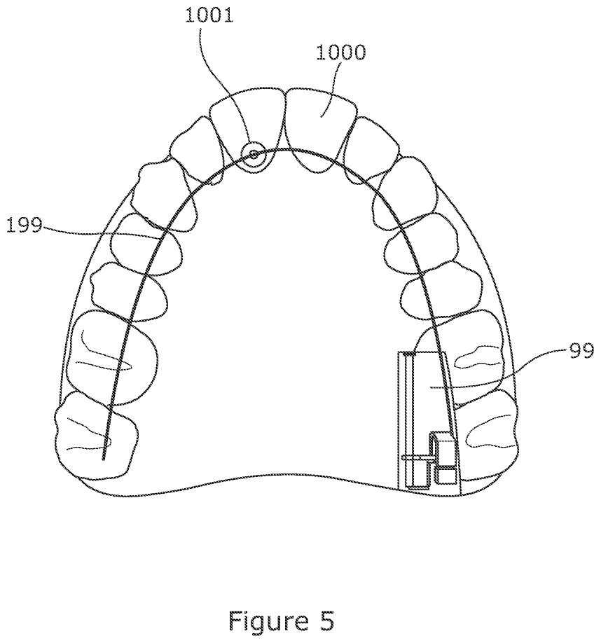

With reference to the figures there is shown an embodiment of the device 99 for apparatus generally comprising one or more wires 199 in use arranged to contact and exert force on one or more teeth 1000, the wire being attached to the teeth 1000 by pads 1001 attached to each tooth (only one pad is illustrated in FIG. 5 for the sake of clarity) and a reel 3, which reel is arranged to move the wire 199.

The device comprises an internal battery for powering a motor and a transceiver is provided to receive a wireless signal which is used to control the motor.

The illustrated embodiment of the device comprises a monolithic housing 1 in which are situated components configured to adjust and hold force on the wire 199 and hence adjust the position of teeth 1000. It is envisaged that housing 1 is mounted inside the patient's mouth in use, and which allows an orthodontist or specialist (referred to hereafter as orthodontist) and patient or user (referred to hereafter as patient) to adjust the force of braces via a software application.

At the instance of first fitting of the device 99, associated wire 199 and pads 1001 the orthodontist develops a treatment plan. Without the device of the invention, the orthodontist would require the patient to be examined in person periodically. Upon any particular examination the orthodontist would decide whether to apply more force to the wire 199 and hence progress the treatment. With the benefit of the invention, the orthodontist sets up a programme of adjustment to the wire 199 that is delivered by the device to a great extent. The orthodontist can monitor the force on the wire 199 at any time. The software may be programmed to make a series of adjustments. For example, the software may be programmed to exert more force on the wire 199 as soon as a teeth movement criteria has been satisfied. Alternatively, the software may be programmed to alert the orthodontist to a teeth movement criteria being satisfied. The orthodontist may then issue a command via the software to cause the force acting on the wire 199 to be increased.

The device includes electronic hardware and the software measures and records progress of movement of the teeth 1000. The gathered information may be used to modify the treatment plan created by the orthodontist, making the process easier, controlled and time effective, both from a patient and orthodontist's perspective.

The illustrated embodiment comprises the following:

Housing 1, in stainless steel, gold-plated. In some embodiments the housing may comprise a plastic area to let the signal of the transceiver out from the module.

Injection moulded reel 3 in Nylon®

Motor 5

Gearbox 9

Brace wire 199 in stainless steel

PCB control processor, UNITFR4 board 6

CR927 battery 8

Battery clip 7

The gold-plated stainless steel of the housing 1 is a non-reactive material that will protect the components inside from acids in food and drink that the patient consumes.

The device in use is mounted securely at the back of the patient's mouth and connects with an internal brace wire 199 that is mounted onto the teeth 1000 typically by means of pads 1001. The device is designed to be non-intrusive and made from a non-reactive material.

The reel 3 is rotatable by the motor to apply force to the wire 199 according to an instruction from at the PCB 6, from the orthodontist or patient.

The battery 8 provides power to the circuit board and the motor. The battery is envisaged to be sized such that it will hold sufficient charge to last the duration of the treatment.

The PCB 6 includes a Bluetooth® transceiver which allows the brace force to be controlled via a software application on a phone or tablet.

The gearbox 9 allows the small motor 5 to apply an amount of force to the brace wire 199 sufficiently large to cause movement of the teeth 1000.

The wire 199 exits the housing at a small exit 2 and a wire gripper 4 provides a lock which prevents the wire from loosening over time and also relieves the motor 5 and gearbox 9 from constant force. The wire gripper 4 may be a cam in some embodiments.

A solenoid may be provided to allow the force of the brace to be released if there is too much discomfort, the solenoid operable to release the wire gripper 4.

The treatment can be started and controlled via the application by the patient and/or the orthodontist after fitting by the orthodontist.

In use:

The specialist takes a precise 3D digital scan of the patient or user's teeth and records the data.

The data is recorded onto the application which provides for the formulation of a treatment plan. Typically, this will be a treatment plan that is the most time efficient and/or the best way of achieving the desired result. It may be envisaged that the method of adjusting the position of teeth 1000 enabled by the device may allow for greater computational accuracy, through accumulation of data for example acquired through a plurality of such devices in use.

The microprocessor is operative to control the motor and at least one sensor senses a characteristic associated with the at least one wire such as sense force, and is operative to send a signal to the microprocessor.

In this way the present invention also provides a system for controlling movement of one or more teeth comprising a device according to any claim and a remote control device which includes a processor operated in accordance with software.

The specialist synchronises the device to the application via Bluetooth®.

The housing 1 of the device 99 is then mounted to the back corner of the user's mouth with the wire 199 fixed to the teeth. The device is configured to be mounted or fixed to teeth or gums with glue or cement in a manner similar to braces, conditioning the enamel before bonding to increase effectiveness of attachment, where pretreatment causes microscopic changes in the enamel, allowing bond materials such as cement or adhesive to flow into the enamel, giving a micro-mechanical attachment.

In some embodiments it may be envisaged that the housing 1 may be mounted using rings or bands to a tooth or teeth. In still further embodiments the housing 1 may be screwed into teeth.

Using the application to start the treatment or after any adjustment in force acting on the wire 199, the orthodontist or patient may override manually the amount of force applied to the wire 199 if there is too much discomfort or pain.

In some embodiments the wire may be envisaged to be attached onto or around teeth or other items, for example which items may be envisaged to be placed against the teeth or active in relation to the positions of the teeth, so as to push or pull against the teeth. In other embodiments the wire may be sprung or biased so as to push the teeth.

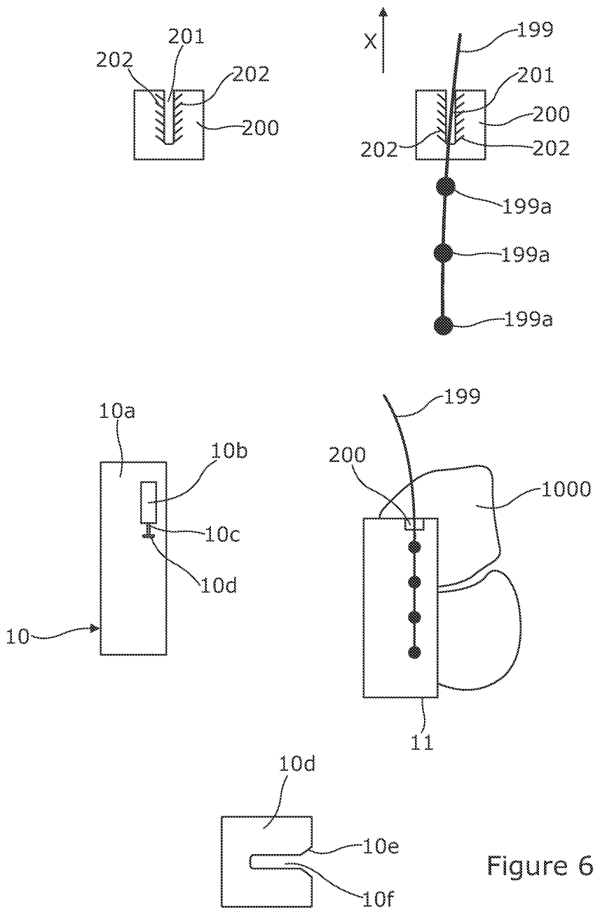

Referring now to FIG. 6, an alternative arrangement is illustrated in which the module 10 is detachably mountable on the teeth 1000 of the user. A mount 11 is attached to the teeth, two in the illustrated example. Typically, the mount would be attached to the teeth 1000 with adhesive or dental cement. The mount 11 remains in place throughout the duration of the treatment. The mount 11 and the module 10 are provided with co-operating parts which allow the module 10 to be attached securely to the mount 11.

The wire 199 is provided with a number of protrusions 199a. When the module 10 is attached to the mount 11, a plate 10d engages with the wire 199, the wire being received in the slot 10f. The plate 10d includes a mouth 10e which guides the wire 199 into the slot 10f as the module 10 is presented up to the mount 11. The plate 10d is attached to the piston 10c of a linear actuator which comprises the piston 10c and a cylinder 10b, the linear actuator being mounted in a body 10a of the module 10. When the piston 10c is extended, the plate 10d engages with one of the protrusions 199a to retract the wire 199. As can be seen from FIG. 6, a non-return mechanism is provided. The non-return mechanism comprises a block 200 with a trough 201 extending therethrough. The side walls of the trough 201 are provided with teeth 202 which are angled so as to resist movement of the wire 199 in the direction X, but permit movement in the direction opposite to direction X. By providing a trough 201, the wire 199 can be engaged therein with the protrusions 199a to one side of the block 200 as shown in FIG. 6. The function of the protrusions is to provide for engagement with the plate 10d of the linear actuator. When the wire 199 is retracted to move the teeth, the wire is held in the new retracted configuration by the teeth 202.

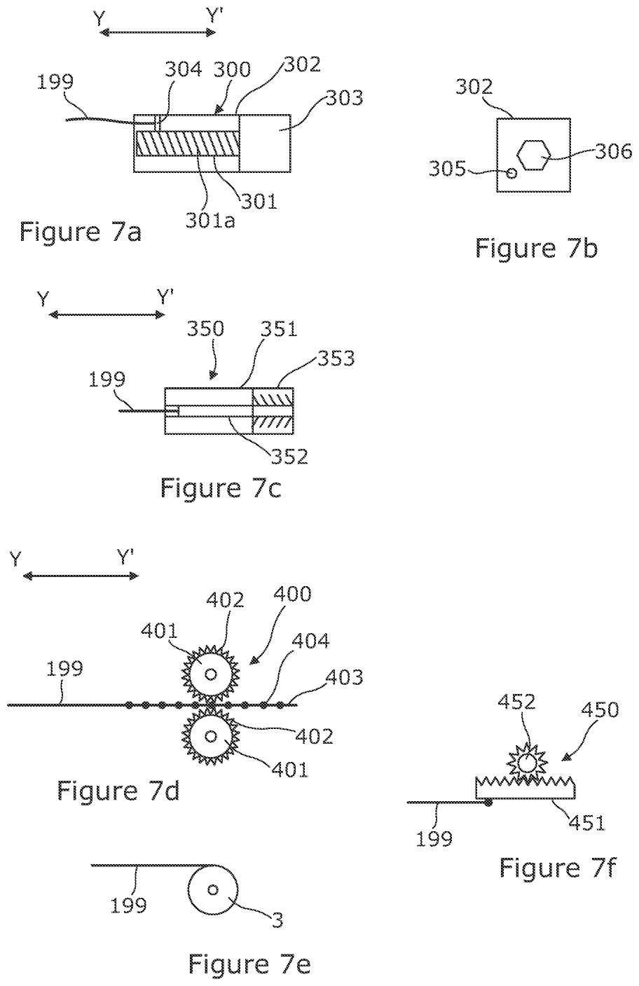

Referring now to FIGS. 7a to 7f, a number of alternative linear mechanisms for retracting or extending the wire 199 illustrated in FIG. 5. Each generates movement in a substantially linear direction.

FIG. 7a illustrates a worm drive 300 comprising a worm shaft 301 which is threaded externally with threads 301a. The worm 301 is mounted in a housing 302 which also mounts a motor 303 to which the worm shaft 301 is attached. A worm follower 304 is also mounted in the housing and engages with threads 301a. The wire 199 is connected to the worm follower 304. The worm follower 304 moves in the direction Y or Y′ depending on the direction of rotation of motor 303. As can be seen from FIG. 7b, the wire 199 passes through a hole 305 in the housing 302. Also shown in FIG. 7b is a manual adjuster in the form of a key 306. The manual adjuster 306 illustrated in this embodiment is operable by a suitably sized Allen key and provides a back up in the event of failure of the motor 303 or the battery powering the motor 303 for example.

FIG. 7c illustrates a linear actuator 350 comprising a housing 351 in which a threaded rod 351 is mounted. A motor 353 is attached to the housing 351. The threaded rod 352 engages with a correspondingly threaded sleeve extending through the centre of the motor 353 so that when the motor 353 is rotated the threaded rod 351, to which the wire 199 is connected, moves back or forth in the direction Y or Y′.

FIG. 7d illustrates a mechanism for retracting or extending the wire 199 which comprises two rollers 401, each of which has circumferentially mounted teeth 402. The wire 199 is attached to a chain 403 which comprises links 404. When the rollers 401 are caused to rotate, each roller being driven to rotate in a direction opposite to the other, the teeth 402 engage with the links 404 of the chain 403 to generate movement of the wire in the direction Y or Y′.

FIG. 7e illustrates a reel 3, the wire 199 being wound around the reel 3. Rotation of the reel 3 generates movement of the wire 199 in the direction Y or Y′, depending on the direction of rotation of the reel.

FIG. 7f illustrates a rack and pinion actuator 450 comprising a rack 451 to which the elongate element 199 is attached and a pinion 452 arranged to mesh with the rack 451. The pinion may be driven by a motor (not shown).

The mechanisms illustrated in FIGS. 7a to 7f are by way of example and are not limitative. Any mechanism capable of adjusting the force on an elongate element and capable of fitting in the space limitations of the users's mouth could be used.

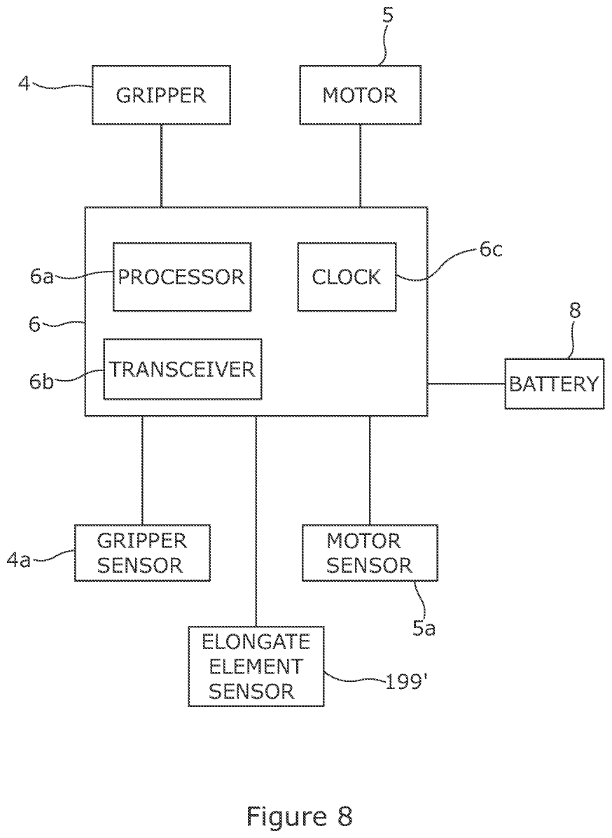

Referring now to FIG. 8, a micro controller is provided by the printed circuit board 6 which includes a processor 6a, a transceiver 6b and a clock 6c. Connected to the printed circuit board 6 are a number of sensor inputs and a number of signal outputs as well as the battery 8 which powers both the components of the printed circuit board 6 an other components requiring electrical power, such as the motor 5.

In normal operation, a command signal is received by the transceiver 6b. The command signal may be to shorten the elongate element by 0.5 mm. The processor sends a signal commanding the motor 5 to turn. When the elongate element sensor 199′ senses that the elongate element has in fact been shortened by 0.5 mm, the elongate element sensor 199′ sends a signal to the processor 6a indicating that the elongate element has been shortened by the desired amount. The processor 6a is programmed with logic causing the signal commanding the motor 5 to turn to be switched to a signal commanding the motor 5 to stop.

When the motor is turning, the motor sensor 5a provides a signal to the processor 6a indicating that the motor is turning, and when the motor ceases to turn, the motor sensor 5a provides a signal indicating that the motor 5 is not turning.

The micro-controller illustrated in FIG. 8 is configured to work with the optional elongate element gripper 4. As described above, the function of the gripper 4 is to hold the elongate element in a particular setting and to relieve the force that would otherwise act on the motor 5.

It is preferred that the gripper 4 grips the elongate element unless a positive force to release the gripper is applied. The processor 6a is programmed with logic such that when the command signal to shorten the elongate element 199 is received by the transceiver 6b, the processor 6a sends a signal to a solenoid associated with the gripper 4 which energises the solenoid to release the grip of the gripper 4 on the elongate element 199. The processor 6a is programmed such that when the motor sensor 5a sends a signal to the processor 6a indicating that the motor 5 is turning, the signal received by the solenoid associated with the gripper 4 remains energised and keeps the gripper off the elongate element 199. When the elongate element 199 has been shortened by the desired amount, the motor stops turning, the motor sensor 5a indicates this. The processor 6a is programmed with logic that causes the solenoid associated with the gripper 4 to be de-energised and the gripper 4 grips the elongate element 199.

The elongate element sensor 5a senses the position and distance moved by the elongate element 199. The signal to shorten the elongate element 199 is received by the transceiver 6b and the signals discussed above are sent, thereby allowing shortening of the elongate element. The sensor 199′ monitors the length of the elongate element as it is being adjusted. When the new length has been reached, the sensors 199′ sends a signal indicating the same. The processor 6a is programmed with logic which causes the motor to stop turning when the elongate element sensor 199′ issues a signal indicating that the new length has been reached.

The clock 6c allows the time period of a whole treatment to be monitored, and also facilitates a set back function. If a set back function is provided, one example of how such function may work would be as follows:

A new length for the elongate element 199 is set. This may cause pain for the patient. The set back function may allow the elongate element to return to its length immediately prior to adjustment. The patient or the orthodontist may set the time period for set back to eight hours, daily for three days following shortening of the elongate element 199. Typically, this would coincide with the time when the patient usually sleeps.

The signal received by the processor 6a via the transceiver would be to release the gripper 4. This in itself may be sufficient to provide the pain relief sought by the patient. Alternatively, and in addition, a signal may be sent to the motor causing the motor to reverse, returning the elongate element 199 to its length immediately prior to shortening. Either the processor 6a is programmed with logic, or a signal is received by the transceiver, such that at the end of the first set back period, measured by the clock 6c, the elongate element 199 is shortened once again as described above. The set back period may be repeated for a number of days.

Instead of adjusting and monitoring the length of the elongate element, the force exerted on the elongate element may be monitored and adjusted.

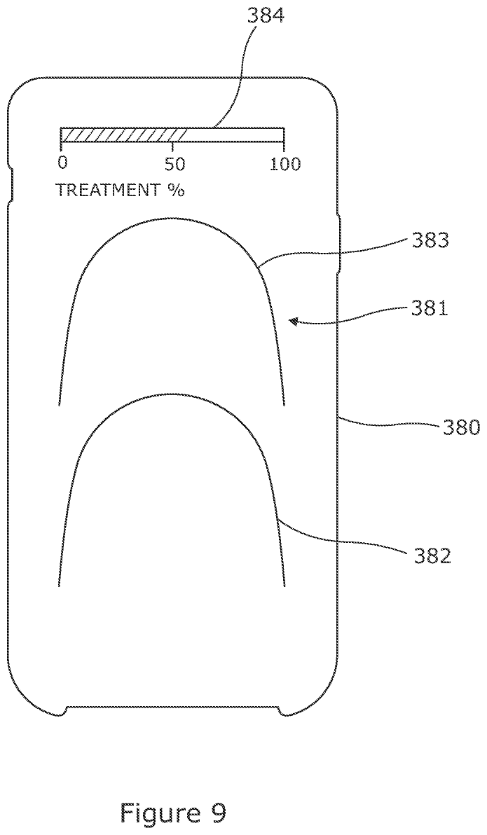

FIG. 9 is an illustration of the imagery that may be displayed on the screen of a computer device, such as a smart phone 380 or other computer, programmed with the software of the invention. The smart phone 380 comprises a display screen 381. An image of the starting shape of the teeth before treatment is shown at 382. The target shape 383 is also shown on the display screen 381 above the starting shape 382. At the top of the display screen 381 is a bar 384 which illustrates in percentage terms how far the treatment has progressed. In the illustration treatment has progressed just beyond 50%.

The images presented on the display screen 381 may depict the starting and end points of a whole treatment programme, or the starting and end points of steps in a programme.

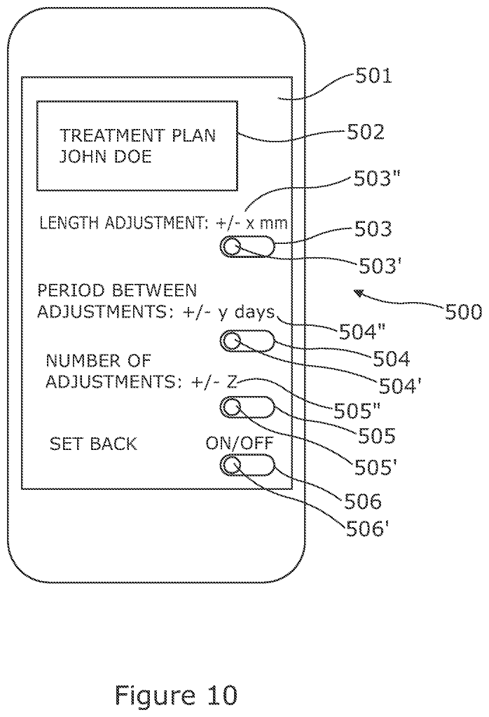

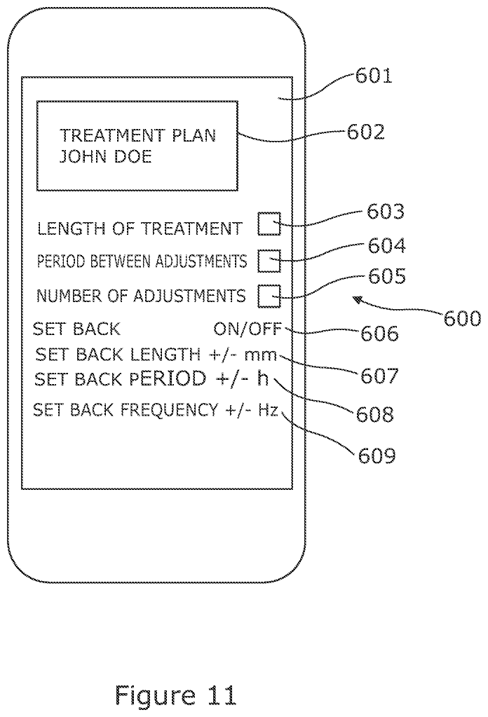

FIGS. 10 and 11 also illustrate a computer device in the form of a smart phone for use by the orthodontist and patient respectively. Referring first to FIG. 10, the smart phone 500 is programmed with the software application which allows the orthodontist to set the parameters of a treatment programme. The parameters that the orthodontist may set are: the length of adjustment, that is the distance the elongate element is moved by at each adjustment; the period between adjustment, that is the number of days between any two adjustments; and the number of adjustments.

As can be seen from the Figure, the smart phone comprises a screen 501 provides various data input possibilities. The name of the patient can be entered in the box 502. The box 503 allows the length of each adjustment to be selected. By touching the button 503′ and moving his finger to the right the orthodontist switches the length adjustment on. The orthodontist may then increase or decrease the length×mm of adjustment using the +/− function 503″.

The period (y days) between adjustments is set by the orthodontist first touching the button 504′ with his finger and swiping to the right. The orthodontist may then increase or decrease the period between adjustments using the +/− function 504″.

The number of adjustments (Z) is set in a similar manner. The orthodontist touches the button 505′ and swipes to the right. The number of adjustments is set using the +/− function 505″.

A set back function is also provided. This allows the patient to relax the force on the elongate element. By swiping the button 506′ in the box 506 to the right the setback function is switched off.

FIG. 11 illustrates the user's smart phone 600. The smart phone 600 includes a screen which provides indications of the patient receiving treatment in box 602, the length of treatment in box 603, the period between treatments in box 604, the tube rod adjustments in box 605 and a set back on/off function with on/off switch 606. For the user to be able to switch the set back function on, the orthodontist must have switched the set back function on in the treatment plan.

When switched to ‘on’ the set back function allows the patient to select the distance by which the elongate element is relaxed with +/− function 607, the set back period with +/− function 608 and the set back frequency with +/− function 609. The software application may be programmed with maximums for each set back parameter. For example, the set back length may be set such that the elongate element cannot be relaxed to a length that is greater than the length prior to the current adjustment. The set back period may be set to a maximum of a part of a day, for example 12 hours. Typically, the frequency of the set back would be daily, for example overnight, for a few days after an adjustment. The patient switches the set back function off when he no longer feels the need to relax the elongate element. The set back frequency should not be short and repetitive as that would loosen the tooth/teeth.

The orthodontist and user my be able to switch between the screens shown in FIGS. 8, 10 and 11 respectively. The software application may also provide a home screen (not shown).

The invention has been described by way of examples only and it will be appreciated that variation may be made to the above-mentioned embodiments without departing from the scope of invention as defined by the claims, in particular but not solely combination of features of described embodiments.

1. A dental device for location in a user's mouth, the mouth having a plurality of teeth therein, the device comprising at least one elongate element, the at least one elongate element arranged to exert a force on at least one of the teeth and a means for adjusting the position of the at least one elongate element in order to manipulate the at least one tooth, and wherein the means for adjusting the position of the at least one elongate element comprises a motor, the motor configured to power the means for adjusting the position of the at least one elongate element.

2. A dental device according to claim 1, wherein the at least one elongate element is selected from the group comprising: a wire; a band; a chain; a coil spring and a ligature.

3. A dental device according to claim 1, wherein the means for adjusting the position of the at least one elongate element is selected from the group comprising: a linear actuator; a lever and a reel.

4. A dental device according to claim 3, wherein the linear actuator is a selected one of: a piston and cylinder; a rack and pinion; a worm drive; and contra-rotating rollers engaging the elongate element therebetween.

5. A dental device according to claim 1, wherein the at least one elongate element is arranged to exert a force on said at least one of the teeth by one of: contacting said at least one of the teeth; and engaging with a member attached to said at least one of the teeth.

6. A dental device according to claim 5, wherein the member attached to said at least one of the teeth is a selected one of: a pad; a bracket and a band.

7. A dental device according to claim 1, wherein the at least one elongate element is provided by a plurality of elongate elements.

8. A dental device according to claim 1, wherein the at least one elongate element engages in an elongate element movement module.

9. A dental device according to claim 8, wherein the module includes a transceiver for wireless connection to a remote controller.

10. A dental device according to claim 8, wherein the module comprises a socket, the socket providing for a wired connection.

11. A dental device according to claim 1, further comprising a source of electrical energy.

12. A dental device according to claim 1, wherein the device comprises at least one sensor, the at least one sensor configured to monitor a condition associated with one of: the at least one elongate element; and said at least one of the teeth.

13. A dental device according to claim 12, wherein the at least one sensor is configured to sense the force exerted on a selected one of: the at least one elongate element; at least one of the teeth; the distance moved by the at least one elongate element ; and the distance moved by at least one of the teeth.

14. A dental device according to claim 1, wherein the device includes a processor configured to one of: control and recognise movement of one of: said at least one of the teeth; and the at least one elongate element.

15. A dental device according to claim 14, wherein the processor is comprised in a micro-controller.

16. A dental device according to claim 14, wherein the processor includes a timer, and wherein the module is configurable to adjust the force acting on the at least one elongate element.

17. A dental device according to claim 1, further including one of: a force release and a force reduction means.

18. A dental device according to claim 1, wherein the said one of: a force release and a force reduction means, is commanded by the processor.

19. A dental device according to claim 1, further comprising an elongate element lock.

20. A dental device according to claim 19, wherein the elongate element lock includes an elongate element gripper.

21. A dental device according to claim 1, wherein the motor is provided with a clutch mechanism.

22. A dental device according to claim 8, wherein the module is adapted for fixed attachment to said at least one of the teeth.

23. A device according to claim 8, wherein the module is adapted for removable attachment to said at least one of the teeth, the said at least one of the teeth having a bracket fixed thereto, the module removably attachable to the bracket.

24. Apparatus for controlling movement of at least one tooth in a user's mouth comprising a dental device comprising at least one elongate element, the at least one elongate element arranged to exert a force on said at least one of the teeth and a means for adjusting the position of the at least one elongate element in order to manipulate the at least one tooth, and wherein the means for adjusting the position of the at least one elongate element comprises a motor, the motor configured to power the means for adjusting the position of the at least one elongate element, a computer device programmed with a software application and signal transmission means providing for the transmission of signals between the computer device and the dental device.
